(19) United States
(12) Patent Application Publication
(10) Pub. No.: US 2007/0156115 A1
(43) Pub. Date: Jul. 5, 2007

(54) INSERTION SECTION FLEXIBLE TUBE AND AN ENDOSCOPE EQUIPPED WITH THE FLEXIBLE TUBE

(75) Inventors: Masayoshi HOSOI, Tokyo (JP); Riichiro TAKESHITA, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/565,145

(22) Filed: Nov. 30, 2006

(30) Foreign Application Priority Data
Dec. 1, 2005 (JP) 2005-348545

(51) Int. Cl. A61M 25/00

(52) U.S. Cl. 604/525

(57) ABSTRACT

A insertion section flexible tube for use in an insertion section of an endoscope includes a flexible tube portion which includes a first core member and a first outer cover which covers the first core member, a bendable portion which includes a second core member and a second outer cover which covers the second core member, the bendable portion having a base end coupled to a tip end of the flexible tube portion; a tightening string for tightening a tip end part of the first outer cover and a base end part of the second outer cover from the outer surfaces thereof; and an adhesive agent for covering and securing the tightening string, wherein at least an outer surface and its vicinity of the first outer cover is formed of a constituent material having low adhesiveness with the adhesive agent, and a region of the outer cover which is covered by the adhesive agent has been subjected to a primary treatment with a primary treatment agent for enhancing the adhesiveness of the outer cover with respect to the adhesive agent.

### BACKGROUND OF THE INVENTION

1. Field of the Invention

The present invention relates to an insertion section flexible tube and an endoscope equipped with the flexible tube, and in particular relates to an insertion section flexible tube for use in an insertion section of an endoscope and an endoscope equipped with the insertion section having the insertion section flexible tube.

2. Description of the Related Art

In the medical fields, an endoscope is used for examining and diagnosing gastrointestinal tract or the like of a patient. In such examination using the endoscope, it is necessary to insert an insertion section of the endoscope (hereinafter, simply referred to as “flexible tube for an endoscope” on occasions) into a deep part of a body cavity of a patient such as stomach, duodena, small intestine, large intestine or the like.

The insertion section includes an insertion section flexible tube which constitutes a main part of the endoscope and a rigid portion provided at the distal or tip end of the insertion section flexible tube for housing an optical system of the endoscope therein.

The insertion section flexible tube has an elongated tubular core member and an outer cover which covers the elongated tubular core member. The tip end portion of the outer cover is tightened with a tightening string to secure the tip end of the outer cover onto the core member, and the tightening string is covered with an adhesive agent. By applying the adhesive agent over the tightening string in this manner, the liquid-tightness (sealability) of the flexible tube is secured to prevent a liquid such as a body fluid from entering into the inside of the endoscope (see, for example, JP-A 2003-126023).

However, depending on the kind of a constituent material of the outer cover, there is a case that sufficient adhesiveness cannot be obtained between the outer cover and the adhesive agent. In such a case, the liquid-tightness of the flexible tube is apt to be lowered. In particular, this tendency becomes conspicuous when a constituent material having low adhesiveness with the adhesive agent (hereinafter, referred to as “low adhesiveness constituent material”) is used as the constituent material of the outer cover.

### SUMMARY OF THE INVENTION

In view of the above problem, an object of the present invention is to provide an insertion section flexible tube for an endoscope having an outer cover formed of a low adhesiveness constituent material but an adhesive agent being capable of adhering to the outer cover with high adhesiveness, to thereby accomplish high liquid-tightness of the insertion section flexible tube.

Another object of the present invention is to provide an endoscope equipped with such an insertion section flexible tube.

In order to achieve the above object, the present invention is directed to an insertion section flexible tube, which comprises: a flexible tube portion which includes a first core member and a first outer cover which covers the first core member, the flexible tube portion having a tip end and the first outer cover having a tip end part; a bendable portion which includes a second core member and a second outer cover which covers the second core member, the second outer cover having a base end part, the bendable portion having a base end which is coupled to the tip end of the flexible tube portion; a tightening string for tightening the tip end part of the first outer cover and the base end part of the second outer cover from the outer surfaces thereof; and an adhesive agent for covering and securing the tightening string, wherein at least an outer surface and its vicinity of the first outer cover is formed of a constituent material having low adhesiveness with the adhesive agent, and a region of the outer cover which is covered by the adhesive agent has been subjected to a primary treatment with a primary treatment agent for enhancing the adhesiveness of the outer cover with respect to the adhesive agent.

According to the present invention having the above structure, it is possible to obtain an insertion section flexible tube having an outer cover formed of a low adhesiveness constituent material but an adhesive agent being capable of adhering to the outer cover with high adhesiveness, to thereby accomplish high liquid-tightness of the insertion section flexible tube.

In the insertion section flexible tube according to the present invention, it is preferred that the primary treatment is a treatment for penetrating the primary treatment agent into the first outer cover from the outer peripheral surface thereof to a predetermined depth in a thickness direction of the outer cover.

According to this primary treatment, since the primary treatment agent is penetrated into the first outer cover to a predetermined depth (thickness) thereof, the primary treatment agent is hard to be peeled off from the first outer cover. Therefore, it is possible to prevent the effect of the primary treatment from being decreased or disappeared, that is, it is possible to keep the effect of the primary treatment for a long period of time. Accordingly, it is possible to prevent peeling-off from occurring at the boundary between the first outer cover and the adhesive agent and thereby keeping the liquid-tightness of the insertion section flexible tube reliably for a long period of time even if the insertion section flexible tube is repeatedly exposed to the sever environment such as sterilization and disinfection treatments.

Further, in the insertion section flexible tube according to the present invention, it is also preferred that the predetermined depth is greater than 30% of the thickness of the first outer cover.

By penetrating the primary treatment agent into the first outer cover sufficiently as described above, the treated region of the first outer cover can be subjected to the primary treatment uniformly.

Furthermore, in the insertion section flexible tube according to the present invention, it is also preferred that the low adhesiveness constituent material contains polyolefin as its main component and the primary treatment agent contains halogenated polyolefin as its main component.

Since halogenated polyolefin is penetrated into the first outer cover, the surface free energy of the first outer cover is lowered and therefore the hydrophobic property, that is, the wettability against the adhesive agent is increased. As a result, the adhesion between the first outer cover and the adhesive agent is enhanced, thereby enabling the liquid-tightness of the insertion section flexible tube to be improved.

In the insertion section flexible tube described above, it is preferred that the halogenated polyolefin is chlorinated polyolefin.

Since chlorinated polyolefin is easily available and chemically stable, it is preferably used as the halogenated polyolefin.

Further, in the insertion section flexible tube described above, it is also preferred that the primary treatment is a treatment for modifying the outer surface of the first outer cover.

According to this primary treatment, the property of the outer surface of the first outer cover is altered or modified from the state that exhibits poor adhesiveness with the adhesive agent (that is, “low adhesiveness property”) to the state that exhibits excellent adhesiveness with the adhesive agent, the adhesive agent can adhere to the first outer cover directly with increased adhesiveness without using any primary treatment agent. Therefore, it is possible to enhance the adhesiveness of the adhesive agent to the first outer cover.

In the insertion section flexible tube described above, it is preferred that the low adhesiveness constituent material contains a fluoro-based resin as its main component and the primary treatment agent contains an aromatic alkali metal compound as its main component.

Such an aromatic alkali metal compound contains an alkali metal having high reactivity, and the alkali metal is reacted with fluorine atom existing one the surface of the first outer cover, so that the fluorine atom is abstracted from the fluoro-based resin and instead thereof a functional group having high hydrophilic property such as a hydroxyl group is introduced, that is, the property of the outer surface of the first outer cover is modified. Accordingly, the treated region of the first outer cover is imparted with high hydrophilic property against the adhesive agent. As a result, it is possible to enhance the adhesion between the first outer cover and the adhesive agent, thereby enabling to improve the liquid-tightness of the insertion section flexible tube.

Further, in the insertion section flexible tube described above, it is preferred that the aromatic alkali metal compound is sodium-naphthalene.

Because sodium-naphthalene has extremely high reactivity with the fluoro-based resin and thus possesses an excellent characteristic for abstracting a fluorine atom from the fluoro-based resin, it is possible to modify the treated region of the first outer cover more reliably.

Another aspect of the present invention is directed to an endoscope equipped with the insertion section flexible tube as described above.

Such an endoscope can have excellent reliability.

These and other objects, structures and results of the present invention will be apparent more clearly when the following detailed description of the preferred embodiment is considered taken in conjunction with the appended drawings.

### BRIEF DESCRIPTION OF THE DRAWINGS

FIG. 1 is an overall view showing an embodiment wherein an endoscope according to the present invention is applied to an electronic endoscope (electronic scope).

FIG. 2 is a partially cross-sectional view showing an insertion section provided in the electronic endoscope of FIG. 1.

FIGS. 3(a) to 3(c) are illustrations (partially cross-sectional views) for explaining a method of manufacturing the insertion section shown in FIG. 2.

### DETAILED DESCRIPTION OF THE PREFERRED EMBODIMENTS

In conjunction with preferred embodiment shown in the accompany drawings, an insertion section flexible tube and an endoscope equipped with the insertion section flexible tube according to the present invention will be described in detail hereinbelow.

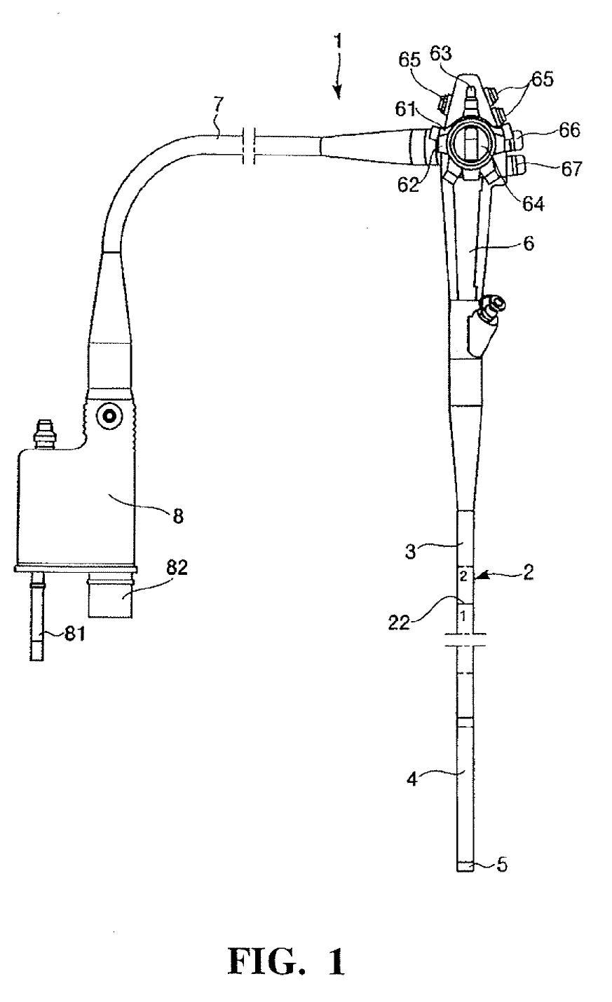

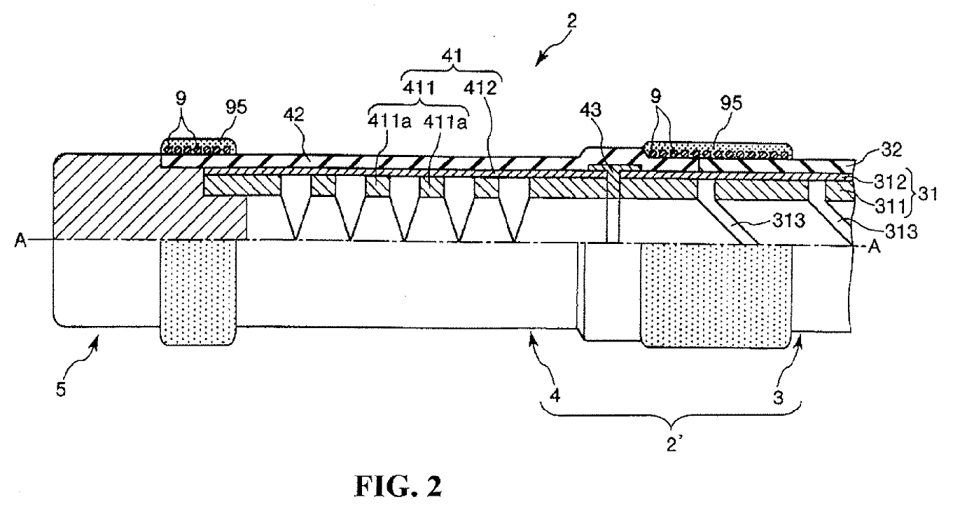

FIG. 1 is an overall view showing an embodiment wherein an endoscope according to the present invention is applied to an electronic endoscope (electronic scope), and FIG. 2 is a partially cross-sectional view showing an insertion section provided in the electronic endoscope of FIG. 1. In this connection, it should be appreciated that, in the following description, the upper side and the lower side in FIG. 1 will be referred to as “base end (proximal end)” and “tip end (distal end)”, respectively, and the right side and the left side in FIG. 2 will be referred to as “base end (proximal end)” and “tip end (distal end)”, respectively.

An electronic endoscope 1 shown in FIG. 1 includes an elongated insertion section 2 having a prescribed flexibility (bendability), an operating section 6 connected to the base end of the insertion section 2 so that it can be grasped by an operator to manipulate the entirety of the electronic endoscope 1, a connecting section flexible tube 7 connected to the operating section 6, and a light source plug 8 provided on the tip end of the connecting section flexible tube 7.

When in use, the insertion section 2 is adapted for insertion into, e.g., a tubular cavity (body cavity) of a living body (patient). As shown in FIG. 1, the insertion section 2 includes an insertion section flexible tube 2′ (an insertion section flexible tube of the present invention) which is comprised of a flexible tube portion 3 and a bendable portion 4 provided on the tip end of the flexible tube portion 3 for bending manipulation, and a rigid portion 5 provided on the tip end (distal end) of the bendable portion 4. These components are arranged in this order from the base end of the insertion section 2.

Each of the flexible tube portion 3 and the bendable portion 4 has an internal space in which various components such as an optical fiber, image signal cables, various tubes and the like (not shown in the drawings) are disposed and inserted.

As shown in FIG. 2, the flexible tube portion 3 is provided with a core member (first core member) 31 and an outer cover (first outer cover) 32 which covers an outer periphery of the core member 31. Further, the bendable portion 4 is provided with a core member (second core member) 41 and an outer cover (second outer cover) 42 which covers an outer periphery of the core member 41.

In a state that the tip end of the core member 31 is coupled to the base end of the core member 41 and the tip end of the outer cover 32 is in contact with the base end of the outer cover 42, the coupling portion between the outer cover 32 and the outer cover 42 is tightened with a tightening string 9, and the tightening string 9 is covered with an adhesive agent 95 to thereby be fixed onto the outer covers 32 and 42.

The core member 31 is comprised of a helical tube 311 and a lattice tube (braided member) 312 which is provided over an outer periphery of the helical tube 311, so that they provide an elongated tubular configuration as a whole.

The helical tube 311 is formed by winding a thin band-shaped member into a spiral shape of uniform diameter, with a spacing 313 left between adjoining turns of the band-shaped member. Preferably, stainless steel, copper alloy or the like may be used as a material for the band-shaped member.

The lattice tube 312 is formed by braiding a plurality of bundles made of metallic or non-metallic fine wires. Preferably, stainless steel, copper alloy or the like, for example, may be used as a material for the fine wires. Furthermore, at least one of the fine wires forming the lattice tube 312 may be coated with a synthetic resin, although not shown in the drawings.

The entire parts of the core member 31 except for the both ends thereof are covered with the outer cover 32. The outer cover 32 is mainly formed of a low adhesiveness resin material which has low adhesiveness with the adhesive agent 95. In this regard, it is to be noted that the outer cover 32 may be formed into a laminated structure comprised of a plurality layers in which only the outermost layer is formed of a low adhesiveness resin material.

Examples of such a low adhesiveness resin material include hydrophobic resins such as polyolefin, fluoro-based resin, silicone-based resin, acrylic resin, polystyrene, polyester, and the like. Among these low adhesiveness resin materials, resin materials containing polyolefin or fluoro-based resin as a main component thereof is preferably used. These resin materials are preferably used as a constituent material of the outer cover 32, since they have excellent chemical resistance and heat resistance as well as suitable elasticity or flexibility. In particular, when the outer cover 32 has suitable elasticity, the tightening string 9 bites into the outer cover 32, thereby enabling the outer cover 32 to be fixed onto the core member 31 more firmly.

Examples of polyolefin include polyethylene, polypropylene, ethylene-propylene copolymer, and ethylene-vinyl acetate copolymer (EVA), and they can be used alone or in a combination of two or more of them.

Examples of fluoro-based resin include polytetrafluoroethylene (PTFE), tetrafluoroethylene-perfluoroalkylvinylether copolymer (PFA) fluorinated-ethylene-propylene copolymer (FEP), ethylene-tetrafluoroethylene copolymer (ETFE), polychlorotrifluoroethylene copolymer (PCTFE), ethylene-chlorotrifluoroethylene copolymer (ECTFE), polyvinylidenefluoride (PVDF), polyvinylfluoride (PPVF or PVF), and the like, and these materials can be used alone or in combination of two or more of them.

The outer cover 32 has a region to be coated or covered by the adhesive agent 95 (hereinafter, referred to as “a treated region” on occasions), and the treated region is subjected to primary treatment by a primary treatment agent for improve adhesiveness with the adhesive agent 95. By this primary treatment, it is possible to improve adhesiveness between the outer cover 32 and the adhesive agent 95, thereby enabling the liquid-tightness of the insertion section 2 to be improved.

The details of the primary treatment will be described later.

The average thickness of each of the outer cover 32 and the outer cover 42 (described later) is not limited to a specific value so long as elements or components disposed inside the flexible tube portion 3 and the bendable portion 4 can be protected by the outer covers 32 and 42 and the flexibility and bendability of the flexible tube portion 3 and the bendable portion 4 are not impaired, but preferably the average thickness should lie in the range of 100 to 3000 μm, and more preferably in the range of 200 to 1000 μm.

Referring back to FIG. 1, the flexible tube portion 3 is provided at its outer surface with scales 22 for indicating the depth that the flexible tube portion 3 is inserted into the body cavity. This allows an operator to manipulate the insertion section 2 while looking the scales 22, when the insertion section 2 is inserted into the body cavity. It is therefore possible to surely guide the tip end of the insertion section 2 to a desired position.

The core member 41 of the bendable portion 4 is comprised of a nodal ring assembly 411 and a lattice tube 412 enclosing an outer periphery of the nodal ring assembly 411 so that they form an elongated tubular configuration as a whole.

The nodal ring assembly 411 is comprised of a plurality of nodal rings 411a with a cross-section of generally annular shape. These nodal rings 411a are disposed side by side along a center axis A of the nodal ring assembly 411. In this nodal ring assembly, the mutually adjacent nodal rings 411a are joined to each other by means of a rivet (not shown in the drawings) and can make an inclined movement with respect to one another. Stainless steel, copper alloy or the like may be preferably used as a material for the nodal rings 411a.

Further, wire guides (not shown) are provided on predetermined nodal rings 411a which are positioned on every predetermined numbers of nodal rings 411a. A rigid portion 5 described below is connected to the wire guides, and bendable portion operating wires, each of which continually extends within the bendable portion 4 and the flexible tube portion 3, are inserted through the wire guides. The bendable portion operating wires are provided, for example, in two sets each including a pair of wire strands. By pulling or releasing each of the bendable portion operating wires, the bendable portion 4 is bent in an arbitrary direction in accordance with the inclined movement of the nodal rings 411a.

At this time, the bendable portion operating wires are supported by the wire guides in such a manner that they can move toward or away from the tip end and the base end.

The outer periphery of the nodal ring assembly 411 is covered with a lattice tube 412 that has the same configuration as the lattice tube 312 described above.

The base end of the core member 41 is connected through a connection tube 43 to the tip end of the core member 31 of the flexible tube portion 3.

The outer periphery of the core member 41 is covered with the outer cover 42 beyond the opposite ends of the core member 41.

The outer cover 42 is mainly formed of a rubber material. Examples of such a rubber material include, but are not particularly limited thereto, butadiene-based rubber such as natural rubber (NR), isoprene rubber (IR), butadiene rubber (BR, 1,2-BR) and styrene butadiene rubber (SBR); diene-based special rubber such as chloroprene rubber (CR) and butadiene-acrylonitrile rubber (NBR); olefin-based rubber such as butyl rubber (IIR), ethylene-propylene rubber (EPM, EPDM), acrylic rubber (ACM, ANN) and halogenated butyl rubber (X-IIR); urethane-based rubber such as urethane rubber (AU, BU); ether-based rubber such as hydrin rubber (CO, ECO, GCO, EGCO); polysulfide-based rubber such as polysulfide rubber (T); silicone rubber (Q), fluoro rubber (FKM, FZ); and chlorinated polyethylene (CM). One of these materials may be used independently or two or more of these materials may be used in combination.

In particular, it is preferred that the outer peripheral surface of the outer cover 42 (in the case of the outer cover having the laminated structure, the outer most layer) is constituted from a rubber material containing fluoro rubber as its main component. The fluoro rubber has excellent chemical resistance and heat resistance, it is possible to prevent deterioration of the outer cover 42 preferably even if the electronic endoscope is repeatedly subjected to a disinfecting or sterilizing treatment conducted in an autoclave or the like.

Further, the rigid portion 5 is attached to the tip end of the bendable portion 4. The rigid portion 5 is constructed from a block of cylindrical shape.

Provided inside the rigid portion 5 is an image-taking element (CCD) not shown in the drawings that takes images of an object on the observation site. The image-taking element is connected to an image signal connector 82 provided on the light source plug section 8, by means of an image signal cable (not shown) continuously extending through the insertion section flexible tube 2′, the operating section 6 and the connecting section flexible tube 7.

Moreover, the rigid portion 5 is joined to the tip ends of the bendable portion operating wires.

Examples of the constituent materials for the rigid portion 5 include, but are not particularly limited to, stainless steel, aluminum, aluminum alloy, titanium and titanium alloy.

A light source connector 81 is provided on the tip end of the light source plug section 8 in parallel with the image signal connector 82. By plugging the light source connector 81 and the image signal connector 82 in a connecting section of a light source processor device (not shown), the light source plug section 8 is connected to the light source processor device. A monitor device (not shown) is connected to the light source processor device via a cable.

The light emitted from the light source processor device is transmitted to the rigid portion 5 through the light source connector 81 and a light guide (not shown) continuously disposed within the light source plug section 8, the connecting section flexible tube 7, the operating section 6 and the insertion section flexible tube 2′. Then, the light is irradiated from the tip end of the rigid portion 5 on a target observation site to illuminate the site. The light guide is comprised of a plurality of bundled light guide members that may be made of, e.g., quartz, multi-component glass, plastic and the like.

The light (object images) reflected from the observation site thus illuminated is sensed by the image-taking element which in turn generates image signals corresponding to the images taken. The image signals are transmitted to the light source plug section 8 through the image signal cable.

And, the image signals are subject to prescribed processing (e.g., signal processing and an image processing) in the light source plug section 8 and the light source processor device, after which the image signals are inputted to the monitor device. The monitor device is adapted to display the images (electronic images) taken by the image-taking element, namely, the motion images monitored by the endoscope.

Further, a first operating knob 61, a second operating knob 62, a first lock lever 63 and a second lock lever 64 are provided on the top surface (in FIG. 1) of the operating section 6 in an independently rotatable manner.

In response to the rotating operation of the operating knobs 61 and 62, the bendable portion operating wires (not shown) disposed within the insertion section flexible tube 2′ are pulled to thereby make the bendable portion 4 bend in four directions, which makes it possible to change the bending direction of the bendable portion 4.

Further, if each of the lock levers 63 and 64 is rotated counterclockwise, it becomes possible to lock (keep) the bendable portion 4 in a bent condition (in up-and-down and right-and-left directions). On the other hand, if each of the lock levers 63 and 64 is rotated clockwise, it becomes possible to release the bendable portion 4 from the locked condition.

A plurality of (three, in the present embodiment) control buttons 65, a suction button 66 and a gas supply/liquid supply button 67 are provided on the side surface or peripheral surface (in FIG. 1) of the operating section 6.

By pushing the control buttons 65 under a state that the electronic endoscope 1 is coupled to the light source processor device (external device), it is possible to remote control various operations of the light source processor device, the monitor device and the like (for example, changing-over of electronic images from motion images to still images and vice versa, starting and/or stopping of an electronic image filing system or an image-taking device, and starting and/or stopping of an electronic image recording device).

The suction button 66 and the gas supply/liquid supply button 67 serve to open and close a suction channel and a gas supply/liquid supply channel (any of them not shown in the drawings), each of which continuously extends through the light source plug section 8, the connecting section flexible tube 7, the operating section 6 and the insertion section 2, with one end thereof being opened at the tip end of the insertion section 2 and the other end thereof being opened at the light source plug section 8.

Namely, unless the suction button 66 and the gas supply/liquid supply button 67 are pushed, the suction channel and the gas supply/liquid supply channel remain closed (in a condition permitting no fluid to pass therethrough). On the other hand, if an operator pushes the suction button 66 and the gas supply/liquid supply button 67, the suction channel and the gas supply/liquid supply channel are opened (in a condition permitting fluid flow therethrough).

Further, when the electronic endoscope 1 is in use, a suction means is coupled to the other end of the suction channel and a gas supply/liquid supply means is connected to the other end of the gas supply/liquid supply channel.

Thus, when the suction channel is in the opened condition, body fluid, blood or the like within the body cavity can be sucked up from the distal end of the insertion section 2. Further, when the gas supply/liquid supply channel is in the opened condition, it is possible to supply liquid or gases into the body cavity from the distal end of the insertion section 2.

Hereinbelow, a description will be made with regard to the manufacturing method of the insertion section 2.

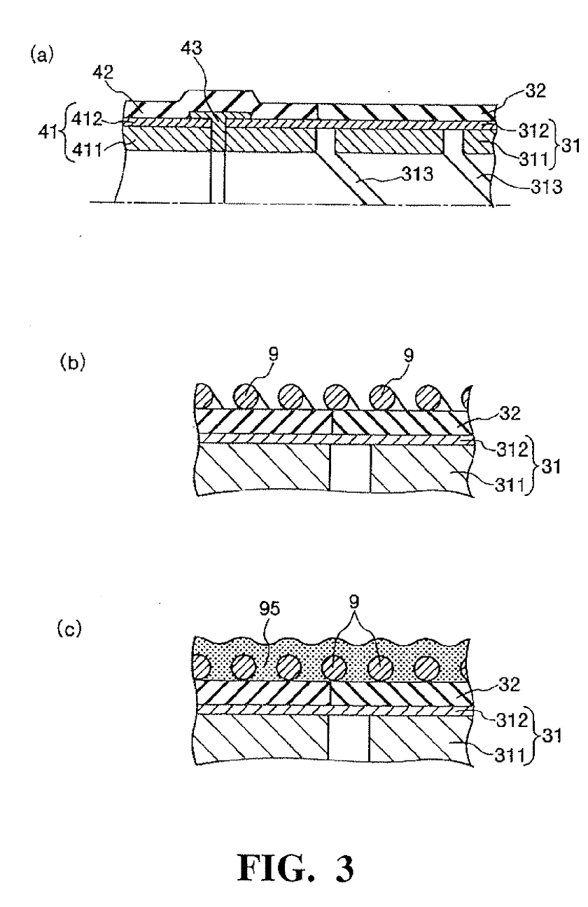

FIGS. 3(a) to 3(c) are illustrations (partially cross-sectional views) for explaining a method of manufacturing the insertion section flexible tube shown in FIG. 2. These figures show the structure of a part of the outer cover and its vicinity in an enlarged manner.

[1] Prepared first are a flexible tube portion 3 that has a core member 31 and an outer cover 32 covering the outer periphery of the core member 31 and a bendable portion 4 that has a core member 41 and an outer cover 42 covering the outer periphery of the core member 41. In addition, a rigid portion 5, a tightening string 9 and an adhesive agent 95 are also prepared.

The average diameter of the tightening string 9 is not particularly limited to a specific value, but preferably in the range of 1 to 500 μm, and more preferably in the range of 10 to 300 μm. If the diameter of the tightening string 9 is too small, a tensioning strength of the tightening string 9 is lowered depending on the kind of the constituent material thereof. In such a case, there is a possibility that it becomes impossible to tighten the outer cover 32 and the outer cover 42 sufficiently to secure or fix the outer cover 32 and the outer cover 42 with respect to the core member 31 and the core member 41. On the other hand, if the diameter of the string 9 is too large, the diameter of the insertion section flexible tube 2′ at the portion tightened by the tightening string becomes too much large, which may result in the case where the burden on a patient is increased when the insertion section 2 is inserted into a body cavity of the patient.

Examples of the adhesive agent 95 include an epoxy-based adhesive agent, an acryl-based adhesive agent, a silicone-based adhesive agent, a polyvinyl acetate-based adhesive agent, a polyvinyl alcohol-based adhesive agent, a polyvinyl acetal-based adhesive, a polyvinyl chloride-based adhesive agent, a polyamide (Nylon)-based adhesive agent, a polyolefin-based adhesive agent, a cellulose-based adhesive agent, a urea-based adhesive agent, a melamine-based adhesive agent, a phenol-based adhesive agent, a resorcinol-based adhesive agent, polyester-based adhesive agent, a polyurethane-based adhesive agent, a polyimide-based adhesive agent, a maleimide-based adhesive agent, a polybenzimidazole-based adhesive agent, α-cyanoacrylate-based adhesive agent, and the like.

Among these adhesive agents, an adhesive agent containing as its main component at least one of epoxy-based adhesive agent, an acryl-based adhesive agent, and silicone-based adhesive agent is preferably used as the adhesive agent 95. Since these adhesive agents are excellent in heat resistance and chemical resistance, it is possible to prevent peeling-off at the boundary between the adhesive agent 95 and the outer covers 32, 42 more reliably. This makes it possible to improve the liquid-tightness of the insertion section flexible tube 2′ (insertion section 2).

[2] Next, at least a region of the outer surface of the outer cover 32 (treated region) to be covered with the adhesive agent 95 is subjected to a primary treatment.

This primary treatment is a treatment for improving adhesiveness between the adhesive agent 95 and the treated region of the outer cover 32 constituted from a low adhesiveness material to which the adhesive agent 95 is difficult to adhere or bond, and this primary treatment is carried out by contacting the treated region to a primary treatment agent.

Generally, the above-mentioned various low adhesiveness materials have excellent heat resistance and chemical resistance. Therefore, by forming at least the outer surface of the outer cover 32 from such a low adhesiveness material, it is possible to obtain an insertion section flexible tube 2′ having sufficient durability even if the insertion section flexible tube 2′ is repeatedly subjected to sterilization and disinfection treatments and thereby it is exposed to chemicals such as various disinfectants and high temperature and high pressure atmosphere many times.

However, in general, there is tendency that the adhesive agent 95 has poor affinity (wettability) to such low adhesiveness materials, and therefore sufficient adhesion against the outer cover 32 can not be obtained.

Therefore, in the present invention, a primary treatment is in advance carried out on the treated region of the outer cover 32 for improving the adhesiveness with the adhesive agent 95 to impart affinity with the adhesive agent 95 to the treated region of the outer cover 32 to thereby improve adhesion between the outer cover 32 and the adhesive agent 95, that is at the boundary therebetween. This makes it possible to prevent peeling-off of the adhesive agent 95 at the boundary during the sterilization and disinfection treatments which are repeatedly carried out. As a result, it is possible to improve the liquid-tightness of the insertion section flexible tube 2′ (insertion section 2) and therefore to enhance the reliability of the electronic endoscope 1.

Examples of such a primary treatment include (I) a treatment for penetrating the primary treatment agent to a certain depth from the outer surface of the outer cover 32, and (II) a treatment for modifying the outer surface of the outer cover 32 using the primary treatment agent.

Hereinbelow, a description will be made with regard to these treatments (I) and (II).

Treatment (I)

According to the treatment (I), since the primary treatment agent is penetrated into the outer cover 32 to a predetermined depth (thickness) thereof, the primary treatment agent is hard to be peeled off from the outer cover 32. Therefore, it is possible to prevent the effect of the primary treatment from being decreased or disappeared, that is, it is possible to keep the effect of the primary treatment for a long period of time. Accordingly, it is possible to prevent peeling-off from occurring at the boundary between the outer cover 32 and the adhesive agent 95 and thereby keeping the liquid-tightness of the insertion section flexible tube 2′ reliably for a long period of time even if the insertion section flexible tube 2′ is repeatedly exposed to the sever environment such as sterilization and disinfection treatments.

The primary treatment agent to be used in this treatment can be appropriately selected depending on the composition of the low adhesiveness material of the outer cover 32. For example, in selecting the primary treatment, affinity to the low adhesiveness material and mutual solubility between these materials may be considered. The following explanation will be made based on the example where the low adhesiveness material contains polyolefin as its main component.

Polyolefin is a polymer which is dominated by C—H bonds. The C—H bond has a relatively low polarizability, and as a result thereof surface free energy of the outer cover 32 indicates a relatively large value. For this reason, the outer cover 32 has hydrophobic property.

As a primary treatment agent used for polyolefin having the above characteristics, a primary treatment agent containing harogenated polyolefin as its main component is preferable. Since the halogenated polyolefin has halogen atoms having a relatively high electronegativity, electrin cloud is biased in its molecular structure, and thus the halogenated polyolefin is polarized at a higher degree as compared to the non-halogenated polyolefin. Since halogenated polyolefin having such characteristics is penetrated into the outer cover 32, the surface free energy of the outer cover 32 is lowered and therefore the hydrophobic property, that is, the wettability against the adhesive agent 95 is increased. As a result, the adhesion between the outer cover 32 and the adhesive agent 95 is enhanced, thereby enabling the liquid-tightness of the insertion section flexible tube 2′ (insertion section 2) to be improved.

Examples of the halogenated atoms include fluorine atom, chloride atom, bromine atom, and iodine atom, but chloride atom is particularly preferable. That is, as halogenated polyolefin, chlorinated polyolefin is preferable. The reason why chlorinated polyolefin is preferable is that it is easily available and chemically stable.

Further, it is preferred that polyolefin used as the low adhesiveness material and halogenated polyolefin used as the primary treatment agent are constituted of the same kind of polyolefin (that is, the same polymer constituted from the same repeated structural units). For example, in the case where the low adhesiveness material is constituted of polyolefin containing as its main component polypropylene, it is preferred that the primary treatment agent is constituted of polyolefin containing as its main component halogenated polyolefin (in particular, chlorinated polyolefin). By using such a combination, it possible to make the primary treatment agent penetrate into the outer cover 32 easily and reliably.

Further, it is preferred that the depth (thickness) to which the primary treatment agent penetrates is 30% or more of the thickness of the outer cover, and more preferably 50% or more. By penetrating the primary treatment agent into the outer cover 32 sufficiently as described above, the treated region of the outer cover 32 is subjected to the primary treatment uniformly.

In this connection, please note that the upper limit value of the ratio of the penetration of the primary treatment agent into the outer cover 32 may be equal to or less than 100% of the thickness of the outer cover 32, but preferably equal to or less than 95% of the thickness of the outer cover 32, and more preferably equal to or less than 90% of the thickness of the outer cover 32. By setting the upper limit value of the ratio of the penetration of the primary treatment agent into the outer cover 32 in this way, it is possible to reliably suppress the flexibility of the flexible tube from being lowered irrespective of the constituent material of the outer cover 32.

Treatment (II)

According to the treatment (II), the property of the outer surface of the outer cover 32 is altered or modified from the state that exhibits poor adhesiveness with the adhesive agent 95 (that is, “low adhesiveness property”) to the state that exhibits excellent adhesiveness with the adhesive agent 95, the adhesive agent 95 can adhere to the outer cover 32 directly with increased adhesiveness without using any primary treatment agent. Therefore, it is possible to enhance the adhesiveness of the adhesive agent 95 to the outer cover 32.

The primary treatment agent to be used can be appropriately selected depending on the composition of the low adhesiveness material used. Hereinbelow, a description will be made based on one example case where the low adhesiveness material is formed of a material containing as its main component a fluoro-based resin.

The fluoro-based resin is a resin which is substantially dominated by C—F bonds. The C—F bond has an especially low polarizability due to specific characteristics possessed by the fluorine molecule, and as a result thereof surface free energy of the outer cover 32 indicates a relatively large value. For this reason, the outer cover 32 has especially high hydrophobic property.

When such fluoro-based resin is used as the low adhesiveness material, a primary treatment agent which contains as its main component an aromatic alkali metal compound is preferably used. Such an aromatic alkali metal compound contains an alkali metal having high reactivity, and the alkali metal is reacted with fluorine atom existing one the surface of the outer cover 32, so that the fluorine atom is abstracted from the fluoro-based resin and in stead thereof a functional group having high hydrophilic property such as a hydroxyl group is introduced, that is, the property of the outer surface of the outer cover 32 is modified. Accordingly, the treated region of the outer cover 32 is imparted with high hydrophilic property against the adhesive agent 95. As a result, it is possible to enhance the adhesion between the outer cover 32 and the adhesive agent 95, thereby enabling to improve the liquid-tightness of the insertion section flexible tube 2′ (insertion section 2).

Examples of the aromatic alkali metal compound include sodium-naphthalene, kalium-naphthalene, kalium-anthracene, and the like, and they can be used independently or in combination of two or more of them. In particular, sodium-naphthalene (Na+[C₁₀H₈]—) is preferable, because sodium-naphthalene has extremely high reactivity with the fluoro-based resin and thus possesses an excellent characteristic for abstracting a fluorine atom from the fluoro-based resin. As a result, it is possible to modify the treated region of the outer cover 32 more reliably.

Further, in the primary treatment (II), it is preferred that the primary treatment agent is removed from the treated region of the outer cover 32 after the primary treatment. This makes it possible to enhance the adhesiveness between the adhesive agent 95 and the outer cover 32.

The method for removing the primary treatment agent is not particularly limited to a specific method, and as one example a method in which the treated region is washed with a cleaning fluid can be mentioned.

Examples of such a cleaning fluid include various organic solvents such as methanol, ethanol, acetone, and the like, various water such as distillated water, purified water, ion-exchange water, reverse osmosis water, and the like.

Further, examples of the method for making the primary treatment agent contact to the treated region of the outer cover 32 in the primary treatment described above include a method in which a predetermined portion (distal end portion) of the outer cover 32 is dipped into a treatment solution containing the primary treatment agent, and a method in which the primary treatment agent is applied onto the treated region of the outer cover 32.

Among these methods, the former method is preferable. According to this method, the treated region of the outer cover 32 can be made contact with the primary treatment agent easily and reliably. In particular, this method is particularly useful for the treatment (I) where the primary treatment agent is penetrated into the treated region of the outer cover 32.

In this case, a liquid (solvent or dispersion medium) used for preparing the treatment liquid can be appropriately selected depending on the composition of the primary treatment agent, and not limited to a specific one. For example, in the case where the primary treatment agent contains as its main component halogenated polyolefin, xylene, toluene, and the like is preferably used. Further, in the case where the primary treatment agent contains as its main component aromatic alkali metal compound, ether such as di ethyl ether, ethyl methyl ether, ethyl vinyl ether, phenetol, diphenyl ether, and the like is preferably used.

In this regard, it is to be noted that various additives such as plasticizer, pigment, and stabilizer (e.g. antioxidant, light stabilizer, antistatic agent, antiblocking agent, and the like) may be added to the treatment liquid, if necessary.

The concentration of the primary treatment agent contained in the treatment liquid is not limited to a specific value, but preferably in the range of 0.5 to 50 parts by weight with respect to 100 parts by weight of the treatment liquid, and preferably in the range of 1 to 40 parts by weight. By setting the concentration of the primary treatment agent to the above-identified range, it is possible to apply a necessary and sufficient amount of the primary treatment agent to the treated region of the outer cover 32.

Further, the time for making the treatment liquid contact with the treated region of the outer cover 32, (that is, the dipping time) is preferably in the range of 10 minutes to 10 hours, and more preferably in the range of 30 minutes to 3 hours. This makes it possible to conduct the primary treatment against the treated region of the outer cover 32 reliably. In this regard, it is to be noted that if the dipping time is longer than the above upper limit value, further effect cannot be obtained.

In the case where the primary treatment agent itself is in a liquid state at room temperature, the primary treatment agent can be used for the treatment liquid as it is.

Further, after making the treatment liquid containing the primary treatment agent contact with the outer cover 32, a dry treatment (that is, a treatment for removing the liquid used for the preparation of the treatment liquid) may be carried out for the treatment liquid as needed.

[3] Then, as shown in FIG. 3(a), the tip end of the core member 31 is coupled (connected) to the base end of the core member 41 through the use of a coupling tube 43, and the base end of the outer cover 42 is made contact with the tip end of the outer cover 32 so that the base end part of the outer cover 42 is placed on the tip part of the core member 31.

Further, the tip end of the core member 41 is made contact with the base end of the rigid portion 5, and the tip end of the outer cover 42 is connected (coupled) to the base end of the rigid portion 5 so that the tip end of the outer cover 42 surrounds the base end of the rigid portion 5 as shown in FIG. 2.

[4] Subsequently, as shown in FIG. 3(b), a connection part between the tip end of the outer cover 32 and the base end of the outer cover 42 are tightened continuously from the outer surface thereof by means of a tightening string 9.

Further, a connection part between the tip end of the outer cover 42 and the base end of the rigid portion 5 is also tightened continuously from the outer surface thereof by means of a tightening string 9.

[5] Next, as shown in FIG. 3(c), an adhesive agent 95 is applied so that the tightening string 9 is covered with the adhesive agent 95, and then the adhesive agent 95 is hardened to affix the tightening string 9.

In this way, the tightening string 9 which tightens the connection part between the tip end of the outer cover 32 and the base end of the outer cover 42 is firmly fixed against the outer cover 32 due to the effect of the primary treatment. With this result, enhanced liquid-tightness can be ensured between the flexible tube portion 3 and the bendable portion 4. Through these steps, it is possible to obtain an insertion section having enhanced liquid-tightness.

In this regard, it is to be noted that an additional step of conducting a surface roughening treatment for roughening at least a region of the outer cover 32 where the primary treatment is to be carried out may be added between the steps [1] and [2] mentioned above. By adding such a surface roughening treatment, a number of irregularities such as fine depressions are formed on the outer peripheral surface of the outer cover 32, so that the adhesive agent 95 can enter the depressions to exhibit an anchor effect, thereby enabling the adhesion between the outer cover 32 and the adhesive agent 95 to be enhanced.

Examples of the surface roughening treatment include a UV irradiation treatment, a plasma irradiation treatment, and a blast treatment, and they can be employed independently or in combination with two or more.

In the foregoing, the insertion section flexible tube and the endoscope equipped with the flexible tube according to the present invention have been described on the basis of the embodiment shown in the drawings. However, the present invention is not limited to the embodiment.

For example, the insertion section flexible tube may be configured so that the base end part of the outer cover 42 is overlapped with the tip end part of the outer cover 32.

Further, the endoscope of the present invention is not limited to an electronic endoscope, and the present invention may be applied to a fiber scope. Further, the endoscope of the present invention is not limited to an endoscope for medical use, and the endoscope of the present invention may be used for an endoscope for industrial use.

### PRACTICAL EXAMPLES

Hereinbelow, practical examples of the present invention will be described in more detail.

1. Production of Electronic Endoscope

In accordance with the following manner, a predetermined number of electronic endoscopes shown in FIG. 1 were manufactured for each of Examples and Comparative Examples.

### Example 1

[1] First, as a low adhesiveness material for use in an outer cover of an insertion section, a tube made of polypropylene (“Santoprene” which is the trademark of Advanced Elastomer Systems Japan Ltd.) was prepared.

[2] Next, a treatment liquid was prepared by dissolving chlorinated polypropylene (primary treatment agent) in a xylene solution (20 Wt %) so that the amount of the chlorinated polypropylene became 30 parts by weight with respect to 100 parts by weight of the treatment liquid.

[3] Next, as shown in FIG. 3(a), the polypropylene tube (hereinafter, simply referred to as “tube”) was dipped or immersed into the treatment liquid for one hour so that the tube was subjected to a primary treatment to thereby obtain an outer cover 32.

Note that when the thus obtained outer tube 32 was observed through its cross section, it has confirmed that the primary treatment agent was penetrated into the outer cover 32 to the depth corresponding to 90% of its thickness.

[4] Next, a rigid portion, a bendable portion, and an flexible tube portion of an endoscope as shown in FIGS. 1 and 2 (“FB-29X” which is a product code of an electronic endoscope of PENTAX Corporation) were prepared.

[5] Next, as shown in FIG. 2, a core member was inserted into the outer cover obtained in the previous step [3] so that the core member was covered by the outer cover, and the tip end of the core member was connected to the base end of the core member of the bendable portion by means of a connection tube.

[6] Next, as shown in FIG. 2, the base end part of the outer cover of the bendable portion was overlaid onto the core member of the flexible tube portion so that the base end of the outer cover of the bendable portion was made contact with the tip end of the outer cover of the flexible tube portion. Further, the tip end part of the outer cover of the bendable portion was overlaid onto the rigid portion.

[7] Next, as shown in FIG. 2, a tightening string having an average diameter of 80 μm was prepared, and the tip end part of the outer cover of the flexible tube portion and the base end part of the outer cover of the bendable portion were tightened continuously from the outer surfaces thereof by means of the tightening string. Further, in the same manner, the tip end part of the outer cover of the bendable portion was also tightened continuously from the outer surface thereof by means of the tightening string.

[8] Next, an adhesive agent (epoxy-based adhesive agent) was applied in such a manner that the tightening string was covered with the adhesive agent, and then the adhesive agent was hardened. In this way, the tightening strings were firmly fixed onto the outer covers with the adhesive agent to thereby obtain an insertion section.

[9] Next, an electronic endoscope was manufactured using the thus obtained insertion section (that is, an insertion section flexible tube of an endoscope).

The details of the constitution of the respective portions were as follows.
Rigid Portion
   Constituent Material: aluminum alloy
   Shape; cylindrical, with three varying outer diameters
   Outer Diameter of Intermediate Part: 9 mm
Bendable Portion
   Dimension of Nodal Ring Assembly: outer diameter of 9 mm and inner diameter of 7 mm
   Constituent Material of Nodal Ring: stainless steel
   Constituent Material of Lattice tube: stainless steel
   Average Thickness of Outer Cover: outer diameter of 10 mm and inner diameter of 9 mm (average thickness of 500 μm)
   Constituent Material of Outer Cover: fluoro-rubber
Flexible Tube Portion
   Dimension of Core Member: outer diameter of 9 mm and inner diameter of 7 mm
   Constituent Material of Helical tube: stainless steel
   Constituent Material of Lattice tube: stainless steel
   Dimension of Outer Cover: outer diameter of 10 mm and inner diameter of 9 mm (average thickness of 500 μm)
   Constituent Material of Outer Cover: polypropylene

### Example 2

An electronic endoscope was manufactured in the same manner as in Example 1, except that the constituent material of the outer cover of the flexible tube portion was changed to polyethylene.

Note that when the thus obtained outer cover was observed through its cross section, it was confirmed that the primary treatment agent was penetrated into the outer cover to the depth corresponding to 95% of its thickness.

### Example 3

An electronic endoscope was manufactured in the same manner as in Example 1, except that the immersion time of the tube into the treatment liquid was changed to 30 seconds so that the primary treatment agent was not penetrated into the tube.

Note that when the thus obtained outer cover was observed through its cross section, it was confirmed that the primary treatment agent was not penetrated into the outer cover at all.

### Example 4

An electronic endoscope was manufactured in the same manner as in Example 1, except that the primary treatment agent was changed to chlorinated polyethylene.

Note that when the thus obtained outer cover was observed through its cross section, it was confirmed that the primary treatment agent was penetrated into the outer cover over the entire of the thickness thereof.

### Example 5

An electronic endoscope was manufactured in the same manner as in Example 1, except that the adhesive agent was changed to acryl-based adhesive agent.

Note that when the thus obtained outer cover was observed through its cross section, it was confirmed that the primary treatment agent was penetrated into the outer cover to the depth corresponding to 90% of its thickness.

### Example 6

An electronic endoscope was manufactured in the same manner as in Example 1, except that the outer surface of the tube was subjected to a blast treatment before the above-described step [3] and that the adhesive agent was changed to a silicone-based adhesive agent.

Note that when the thus obtained outer tube was observed through its cross section, it was confirmed that the primary treatment agent was penetrated into the outer cover to the depth corresponding to 85% of its thickness.

### Example 7

An electronic endoscope was manufactured in the same manner as in Example 1, except that the constituent material of the outer cover of the flexible tube portion was changed to polytetrafluoroethylene and that the primary treatment agent was changed to sodium-naphthalene.

Note that a treatment liquid was prepared by dissolving sodium naphthalene (primary treatment agent) in an ether solution so that the amount of the chlorinated polypropylene became 20 parts by weight with respect to 100 parts by weight of the treatment liquid.

Further, note that when the thus obtained outer tube was observed through its cross section, it was confirmed that the primary treatment agent was not penetrated into the outer cover at all.

### Comparative Example 1

An electronic endoscope was manufactured in the same manner as in Example 1, except that the primary treatment was omitted.

### Comparative Example 2

An electronic endoscope was manufactured in the same manner as in Comparative Example 1, except that instead of using the low adhesiveness material the constituent material of the outer cover of the flexible tube portion was changed to polyurethane having a relatively high adhesiveness with the adhesive agent and that the primary treatment was omitted.

### Comparative Example 3

An electronic endoscope was manufactured in the same manner as in Comparative Example 1, except that the steps [2] and [3] were omitted and a blast treatment was carried out before the step [4].

2. Evaluation

2.1 Evaluation of Adhesiveness

In each of the electronic endoscopes manufactured respectively in the Examples 1 to 7 and the Comparative Examples 1 to 3, the bendable portion was caused to bend by gradually rotating the operating knob until the magnitude of the angular force applied was increased from the practical use range to the out-of-practical use range. Then, for each electronic endoscope after the bending manipulation, observation was made as to whether the adhesive agent that covers the tightening string was peeled off or not (that is, the appearance of the tightened region was observed). The observation results were evaluated in accordance with the following four criteria.

A: No peeling-off of the adhesive agent was observed even if the bendable portion was bent over the practical use range.

B: No problem occurred within the practical use range, but the adhesive agent was peeled off when the bendable portion was bent out of the practical use range.

C: The adhesive agent was slightly peeled off when the bendable portion was bent within the practical use range.

D: The adhesive agent was peeled off when the bendable portion was bent within the practical use range.

2.2 Evaluation of Chemical Resistance

First of all, for each of the fifteen electronic endoscopes manufactured in each of the Examples 1 to 7 and the Comparative Examples 1 to 3, the region tightened by the tightening string was immersed into chemical solution according to the following immersion test conditions.
Immersion Test A
   Ingredient of Chemical Solution: glutaraldehyde
   Content of Ingredient: 3%
   Time Immersed: 30 minutes
   Test Cycle: 5,000 times
   Number of Piece Tested: 5
Immersion Test B
   Ingredient of Chemical Solution: acetyl hydroperoxide
   Content of Ingredient: 0.3%
   Time Immersed; 30 minutes
   Test Cycle: 5,000 times
   Number of Piece Tested: 5
Immersion Test C
   Ingredient of Chemical Solution: hydrogen peroxide
   Content of Ingredient: 30%
   Time Immersed: 60 minutes
   Test Cycle: 5,000 times
   Number of Piece Tested: 5

Subsequently, for each of the respective electronic endoscopes which have undergone the immersion tests, the bendable portion was caused to bend by gradually rotating the operating knob until the magnitude of the angular force applied was increased from the practical use range to the out-of-practical use range.

Then, for each of the respective electronic endoscopes which have undergone the bending operation, observation was made as to whether the adhesive agent that covers the tightening string was peeled off or not to know the chemical resistance. The observation results were evaluated in accordance with the following four criteria.

A: No peeling-off of the adhesive agent was observed even if the bendable portion was bent over the practical use range.

B: No problem occurred within the practical use range, but the adhesive agent was peeled off when the bendable portion was bent out of the practical use range.

C: The adhesive agent was slightly peeled oft when the bendable portion was bent within the practical use range.

D: The adhesive agent was peeled off when the bendable portion was bent within the practical use range.

2.3 Evaluation for Liquid-tightness

For each of the electronic endoscopes manufactured respectively in the Examples 1 to 7 and the Comparative Examples 1 to 3, a disinfecting or sterilizing treatment was repeatedly conducted under high temperature in an autoclave. The conditions of the disinfecting or sterilizing treatment were as follows.

Temperature: 120° C.

Time: 10 minutes

Cycle Repeated: 3,000 times

In this test, upon every 500 cycles, an image produced by each endoscope was checked to confirm as to whether or not there was any trouble in the functions of the electronic endoscope. The results were evaluated in accordance with the following four criteria.

A: No trouble was confirmed even after 3,000 cycles.

B: Any trouble was confirmed after 3,000 cycles.

C: Any trouble was confirmed between 1,500 and 2,500 cycles.

D: Any trouble was confirmed between 0 and 1,000 cycles.

The results of the evaluations made in the above items 2.1 through 2.3 are shown in the following Table 1.

TABLE 1

| | Constructions of insertion section | | | | | | | | | |
|---|---|---|---|---|---|---|---|---|---|---|
| | | Primary treatment | | | | | | | | |
| | | agent | | | | | | | | |
| | | | Degree of | | | | | | | |
| | Constituent | | penetration | | | | | | | |
| | material of | | with | | | | | | | |
| | outer cover | | respect to | | | Results of evaluation | | | | |
| | of flexible | | thickness | | Kind of | | Chemical resistance | | | |
| | tubular | | of outer | Roughening | adhesive | | Immersion | Immersion | Immersion | Liquid- |
| | section | Composition | cover [%] | treatment | agent | Adhesiveness | test A | test B | test C | tightness |
| Ex. 1 | PP | CPP | 90 | — | Epoxy-based | A | A | A | A | A |
| | | | | | adhesive | | | | | |
| | | | | | agent | | | | | |
| Ex. 2 | PE | CPP | 95 | — | Epoxy-based | B | B | C | C | A |
| | | | | | adhesive | | | | | |
| | | | | | agent | | | | | |
| Ex. 3 | PP | CPP | 0 | — | Epoxy-based | B | C | C | C | B |
| | | | | | adhesive | | | | | |
| | | | | | agent | | | | | |
| Ex. 4 | PP | CPE | 100 | — | Epoxy-based | B | B | C | C | A |
| | | | | | adhesive | | | | | |
| | | | | | agent | | | | | |
| Ex. 5 | PP | CPP | 90 | — | Acryl-based | B | B | B | B | A |
| | | | | | adhesive | | | | | |
| | | | | | agent | | | | | |
| Ex. 6 | PP | CPP | 85 | Blast | Silicone- | A | A | A | A | A |
| | | | | treatment | based | | | | | |
| | | | | | adhesive | | | | | |
| | | | | | agent | | | | | |
| Ex. 7 | PTFE | SN | 0 | — | Epoxy-based | A | A | A | A | A |
| | | | | | adhesive | | | | | |
| | | | | | agent | | | | | |
| Comp. Ex. 1 | PP | — | — | — | Epoxy-based | D | D | D | D | D |
| | | | | | adhesive | | | | | |
| | | | | | agent | | | | | |
| Comp. Ex. 2 | PUR | — | — | — | Epoxy-based | B | A | D | C | C |
| | | | | | adhesive | | | | | |
| | | | | | agent | | | | | |
| Comp. Ex. 3 | PP | — | — | Blast | Epoxy-based | C | C | D | D | D |
| | | | | treatment | adhesive | | | | | |
| | | | | | agent | | | | | |

※ PP: Polypropylene
PE: Polyethylene
PUR: Polyurethane
PTFE: Polytetrafluoroethylene
CPP: Chlorinated polypropylene
CPE: Chlorinated polyethylene
SN: Sodium-Naphtalene

As is apparent from the evaluation result of 2.1, in the case of the electronic endoscopes manufactured in each of the Examples 1 to 7, no peeling off could not be recognized at the region tightened by the tightening string even if the bendable portion was bent within the practical use range. Presumably, this is because the adhesiveness between the adhesive agent and the outer cover was sufficiently high so as to be able to endure the practical use.

In contrast, in each of the electronic endoscopes of the Comparative Example 1, the adhesive agent was peeled off only when the bendable portion was slightly bent, while in each of the electronic endoscopes of the Comparative Example 2, no peeling-off of the adhesive agent occurred at the tightening region even if the bendable portion was bent within the practical use range.

Further, as is apparent from the evaluation result of 2.2, the electronic endoscopes manufactured in each of the Examples 1 to 7 almost exhibited high durability against the chemical solution of superior disinfectability and strong sterilizing property, such as glutaraldehyde, acetyl hydroperoxide and hydrogen peroxide.

In contrast, the tightened region of each of the electronic endoscopes manufactured in the Comparative Examples 1 to 3 was degraded by the chemical solution, and the adhesive agent was peeled off from the tightening region by the bending operation with a force in the practical use range. In particular, in each of the electronic endoscopes manufactured in the Comparative Example 1, the adhesive agent was peeled off soon.

Moreover, as is apparent from the evaluation result of 2.3, in each of the electronic endoscopes manufactured in the Examples 1 to 7, any trouble could not be found on the functions thereof even if a disinfecting or sterilizing treatment in an autoclave was repeatedly conducted for 3,000 times.

In contrast, in each of the electronic endoscopes manufactured in the Comparative Examples 1 to 3, a phenomenon that an image produced by the endoscope was blurred has been confirmed. In particular, in each of the electronic endoscopes manufactured in the Comparative Example 1, any trouble could be confirmed in the repeated cycles of several ten times. It is supposed that this phenomenon was caused by loss of cleanness of the imaging element. Further, it is also supposed that the cause of the loss of cleanness was resulted from the fact that water or the like was entered inside the electronic endoscope due to the peeling-off of the adhesive agent by the disinfecting or sterilizing treatment.

Finally, it is to be understood that the present disclosure relates to the subject matter contained in Japanese Patent Application No. 2005-348545 (filed on Dec. 1, 2005) which is expressly incorporated herein by reference in its entirety.

What is claimed is:

1. A insertion section flexible tube, comprising:
a flexible tube portion which includes a first core member and a first outer cover which covers the first core member, the flexible tube portion having a tip end and the first outer cover having a tip end part;
a bendable portion which includes a second core member and a second outer cover which covers the second core member, the second outer cover having a base end part, the bendable portion having a base end which is coupled to the tip end of the flexible tube portion;
a tightening string for tightening the tip end part of the first outer cover and the base end part of the second outer cover from the outer surfaces thereof; and
an adhesive agent for covering and securing the tightening string,
wherein at least an outer surface and its vicinity of the first outer cover is formed of a constituent material having low adhesiveness with the adhesive agent, and a region of the outer cover which is covered by the adhesive agent has been subjected to a primary treatment with a primary treatment agent for enhancing the adhesiveness of the outer cover with respect to the adhesive agent.

2. The insertion section flexible tube as claimed in claim 1, wherein the primary treatment is a treatment for penetrating the primary treatment agent into the first outer cover from the outer peripheral surface thereof to a predetermined depth in a thickness direction of the outer cover.

3. The insertion section flexible tube as claimed in claim 1, wherein the predetermined depth is greater than 30% of the thickness of the first outer cover.

4. The insertion section flexible tube as claimed in claim 1, wherein the low adhesiveness constituent material contains polyolefin as its main component and the primary treatment agent contains halogenated polyolefin as its main component.

5. The insertion section flexible tube as claimed in claim 4, wherein the halogenated polyolefin is chlorinated polyolefin.

6. The insertion section flexible tube as claimed in claim 1, wherein the primary treatment is a treatment for modifying the outer surface of the first outer cover.

7. The insertion section flexible tube as claimed in claim 6, wherein the low adhesiveness constituent material contains a fluoro-based resin as its main component and the primary treatment agent contains an aromatic alkali metal compound as its main component.

8. The insertion section flexible tube as claimed in claim 7, wherein the aromatic alkali metal compound is sodium-naphthalene.

9. An endoscope equipped with the insertion section flexible tube as defined in claim 1.
